(19) United States
(12) Patent Application Publication
(10) Pub. No.: US 2024/0341617 A1
(43) Pub. Date: Oct. 17, 2024

(54) IMPLANTABLE MICRO-COIL RESONATOR FOR DEEP TISSUE ELECTRON PARAMAGNETIC RESONANCE

(71) Applicant: THE UNIVERSITY OF CHICAGO, Chicago, IL (US)

(72) Inventors: Bulent Aydogan, Orland Park, IL (US); Howard Halpern, Chicago, IL (US); Subramanian Sundramoorthy, Chicago, IL (US); Erik Pearson, Chicago, IL (US); Boris Epel, Chicago, IL (US); Daniela Olivera Velarde, Chicago, IL (US); Ann Barry Flood, Lyme, NH (US); Harold M. Swartz, Lyme, NH (US); Wilson Schreiber, West Lebanon, NH (US)

(21) Appl. No.: 18/635,509

(22) Filed: Apr. 15, 2024

Related U.S. Application Data
(60) Provisional application No. 63/496,095, filed on Apr. 14, 2023.

(51) Int. Cl. A61B 5/055; A61B 5/00

(52) U.S. Cl. CPC A61B 5/055; A61B 5/4869; A61B 2562/12; A61B 2562/222; A61B 2562/225

(57) ABSTRACT

An oxygen sensing system includes a coupling loop that connects to an electron paramagnetic resonance (EPR) bridge. The oxygen sensing system also includes a cable connected to the coupling loop, where the cable acts as a transmission line. The oxygen sensing system further includes a plurality of oxygen sensors formed on the cable, where each of the oxygen sensors comprises a multi-turn coil.

### CROSS-REFERENCE TO RELATED APPLICATION

The present application claims the priority benefit of U.S. Provisional Patent App. No. 63/496,095 filed on Apr. 14, 2023, the entire disclosure of which is incorporated herein by reference.

### BACKGROUND

Tumors, which are abnormal masses of tissue that are sometimes cancerous, often grow rapidly in size, as compared to the growth of normal, non-tumorous tissue. This rapid growth can result in a condition called hypoxia when the tumor outgrows its blood supply. Hypoxia refers to having an oxygen concentration that is significantly lower than the oxygen concentrations of healthy tissue. In response to tumor hypoxia and in order to support continuous growth and proliferation, cancer cells have been found to alter their metabolism. This altered metabolism of the cancer cells can lead to an increase in metastatic behavior and resistance to treatment.

### SUMMARY

An illustrative oxygen sensing system includes a coupling loop that connects to an electron paramagnetic resonance (EPR) bridge. The oxygen sensing system also includes a cable connected to the coupling loop, where the cable acts as a transmission line. The oxygen sensing system further includes a plurality of oxygen sensors formed on the cable, where each of the oxygen sensors comprises a multi-turn coil.

In an illustrative embodiment, the plurality of oxygen sensors are connected in parallel. In another embodiment, the cable comprises a twisted pair wire cable. In another embodiment, each oxygen sensor includes a twisted pair tail that extends from the oxygen sensor. In another embodiment, the twisted pair tail varies in length for each of the oxygen sensors. In some embodiments, each multi-turn coil includes microcrystals that are coated with lithium phthalocyanine (LiPc). In another embodiment, a polydimethylsiloxane (PDMS) layer that is molded over the cable and the plurality of oxygen sensors.

In another embodiment, a polytetrafluoroethylene (PTFE) tube covers the plurality of oxygen sensors and at least a portion of the cable. Specifically, the portion of the cable and the plurality of oxygen sensors form an implant portion of the system, and the PTFE tube covers the implant portion. In another illustrative embodiment, the multi-turn coil of each oxygen sensor is oriented axially relative to the cable. The system can also include the EPR bridge, along with a clamp mechanism mounted to an end of the EPR bridge. The clamp mechanism is sized to receive the coupling loop. In one embodiment, the clamp mechanism comprises a clamshell clamp that closes around the coupling loop.

An illustrative method of forming an oxygen sensing system includes forming a coupling loop that is sized to connect to an electron paramagnetic resonance (EPR) bridge. The method also includes mounting a cable to the coupling loop, where the cable acts as a transmission line. The method includes forming a plurality of oxygen sensors on the cable, where each of the oxygen sensors comprises a multi-turn coil.

In an illustrative embodiment, the plurality of oxygen sensors are formed on the cable such that the oxygen sensors are connected in parallel. In another embodiment, the plurality of oxygen sensors are formed such that each oxygen sensor includes a twisted pair tail that extends from the oxygen sensor. In one embodiment, the twisted pair tail varies in length for each oxygen sensor.

The method can also include forming a mixture of lithium phthalocyanine (LiPc) microcrystals and polydimethylsiloxane (PDMS) and loading the mixture into the multi-turn coil to form each oxygen sensor in the plurality of oxygen sensors. At least a portion of the cable and the plurality of oxygen sensors form an implant portion of the system, and the method can also include covering the implant portion with polytetrafluoroethylene (PTFE) tube. The method can also include mounting a clamp mechanism to the EPR bridge, where the clamp mechanism is sized to receive the coupling loop. The method can further include forming the clamp mechanism as a clamshell clamp.

Other principal features and advantages of the invention will become apparent to those skilled in the art upon review of the following drawings, the detailed description, and the appended claims.

### BRIEF DESCRIPTION OF THE DRAWINGS

Illustrative Embodiments of the Invention Will Hereafter be Described with Reference to the Accompanying Drawings, Wherein Like Numerals Denote Like Elements.

FIG. 1A depicts a first (Generation 2) oxygen sensor used in a deep tissue electron paramagnetic resonance system in accordance with an illustrative embodiment.

FIG. 1B depicts a second (Generation 3) oxygen sensor used in a deep tissue electron paramagnetic resonance system in accordance with an illustrative embodiment.

FIG. 1C depicts a plurality of sensors connected in series in accordance with an illustrative embodiment.

FIG. 1D depicts a plurality of sensors connected in parallel in accordance with an illustrative embodiment.

FIG. 1E depicts a first subset of sensors connected in series, a second subset of sensors connected in series, and a parallel connection between the first subset and the second subset in accordance with an illustrative embodiment.

FIG. 1F depicts a sensor system and a coupling mechanism sized to receive a portion of the sensor system in accordance with an illustrative embodiment.

FIG. 1G depicts the sensor system mounted to the coupling mechanism in accordance with an illustrative embodiment.

FIG. 1H depicts a prototype of the proposed sensing system compared to other common fiducials in accordance with an illustrative embodiment.

FIG. 2 depicts relative signal intensity of EPR spectra from a Generation 2 sensor system and the proposed sensor system in accordance with an illustrative embodiment.

FIG. 3 depicts a one-minute EPR spectra from a Generation 2 sensor system and the proposed Generation 3 micro-coil sensor system in accordance with an illustrative embodiment.

FIG. 4 illustrates how the spatially resolved O₂ measurements from a four-point O₂ probe will be obtained in accordance with an illustrative embodiment.

FIG. 5 is a block diagram of a computing system for a deep tissue electron paramagnetic resonance system in accordance with an illustrative embodiment.

### DETAILED DESCRIPTION

Tumor hypoxia is a major adverse factor in cancer and is associated with poor outcomes regardless of treatment modality. It has been demonstrated that the efficacy of radiation therapy is predicted more significantly by oxygen (O₂) levels in the tumor than by stage, tumor morphology, or tumor size. Specifically, there is a long-standing consensus that, if O₂ levels in tumors could be increased at the time of irradiation, there could be very significant improvements in outcomes. To date, however, clinical trials to test the efficacy of O₂-enhancing interventions (such as the Avastin and ARCON trials) have not achieved practice-changing results. In retrospect, the inventors have determined that these failures were due to the inability to know which patients had tumors that responded to O₂-interventions and the time window for the response so that the radiation could be delivered at a time that would improve outcomes.

Through studies and experimentation, the inventors found that some tumors would not be expected to have their response to radiation changed significantly by O₂ interventions, because they either did not respond to breathing O₂-enriched gas or already had high levels of O₂ (i.e., so that they were already not resistant to radiation due to hypoxia). Therefore, clinical studies aiming to evaluate clinical benefit of improving tumor O₂ should have a practical and robust methodology to measure O₂ changes in tumors before (to select patients), after, and during O₂-enriching intervention to determine the treatment window. Oxygen monitoring is also important during the course of cancer therapy to determine if there are changes in the response to the intervention. This would make it possible to have a robust test of the hypotheses that O₂-increasing interventions can improve outcomes. Although there are several clinically applied methods that provide some information on O₂ levels in vivo (particularly F-misonidazole, FMISO-PET and MRI), existing techniques do not provide direct measures of O₂ in tumors and surrounding tissues, nor allow repeated measurements of O₂ at the same site under clinically applicable conditions to follow the time-course of hypoxia.

Currently there are no clinical devices or techniques to monitor tumor oxygen level in real time. Described herein is a new device that will provide spatially distributed, repeatable, and reliable O₂ measurements at any medically relevant depth under clinically useful conditions to optimize therapeutic delivery of radiation. Specifically, the proposed device can be used to provide knowledge about hypoxia levels from interventions and treatment over the course of therapy, which can be used to optimize the timing of radiotherapy fractions to be delivered. The system can also be used to identify patients that will and will not benefit from increased tumor O₂ levels during radiation. This will allow physicians to evaluate and demonstrate, for the first time, improved outcomes that are expected to result from delivering treatment fractions at time(s) when oxygenation is improved in hypoxic tumor regions.

In an illustrative embodiment, the proposed device works in conjunction with Electron Paramagnetic Resonance (EPR) oximetry. The capabilities and feasibility of EPR oximetry have been previously demonstrated with particulate oxygen sensitive EPR materials directly implanted in tissue such as ‘India Ink’ and ‘OxyChips,’ made of paramagnetic particulates encapsulated in a O₂ permeable polymer, polydimethylsiloxane (PDMS). It has been shown that EPR oximetry in preclinical models is a very effective method for direct measurement of the partial pressure of oxygen (pO₂) in tissues repeatedly with the potential to improve the efficacy of the clinical intervention or treatment. While clinical studies using OxyChips implanted directly in tissue have been successfully carried out in some patients, they were successful only for tumors at depths less than 11 millimeters (mm).

Preclinical studies have demonstrated that pO₂ measurements can be made effectively using an implantable resonator containing multiple oxygen sensors (ImR) connected to a spectrometer for EPR signal acquisition via an external surface loop resonator to couple the RF excitation to the ImR. One implementation, which is referred to herein as Generation 1 ImR, includes a thin enameled copper wire structure that has a 1-cm coupling loop and one to four 0.7-mm diameter wire loops containing oxygen sensitive spin probes. The coupling loop and the smaller sensing loops are interconnected by a twisted pair transmission line, enabling measurement at depths beyond the previous 11 mm limit. Additionally, the wire structure enables imaging via fluoroscopy, which does not resolve the individual single-turn sensor loops, but which does enable determination of the implant position.

In another implementation, referred to herein as a Generation 2 ImR, the copper wire was replaced by FDA approved and clinically used, high tensile strength 0.004 inch diameter MP35N wire insulated with PTFE (California Fine Wire Co.). This wire avoids breakages observed when Generation 1 ImRs, using copper wire, were tested in actively moving tissues in animal models. In Generation 2 ImRs, the sensor loops contained the oxygen sensitive paramagnetic material lithium octa-n-butoxynaphthalocyanine (LiNc-BuO) passivated by oxygen-permeable PDMS silicone elastomer. The twisted pair transmission line was terminated with an open end on the coupling loop side. Also, the entire ImR structure was encapsulated in PDMS and a Teflon (PTFE) sleeve. Both PDMS and PTFE are oxygen-permeable and biocompatible materials.

When used in a large animal (pig), Generation 1 ImRs had mechanical problems that resulted in the paramagnetic materials being dislodged from the sensing loops and/or failures due to work-hardened broken wires. This was partially addressed in the most recent versions, enabling measurements of pO₂ at sites as deep as 2 cm in rabbits. While replacing the copper wire with MP35N improved tensile strength of the ImR, it resulted in a decrease in signal due to the relatively higher resistance of MP35N wire compared to copper wire for a given size. The specific resistivity of the MP35N wire used is 103.3 μΩ-cm, compared to 1.68 μΩ-cm of the previously used copper. As a result, the signal was almost lost when the single-turn loops were located at the end of a 15-cm long transmission line in a Generation 2 ImR inside a nitrogen box.

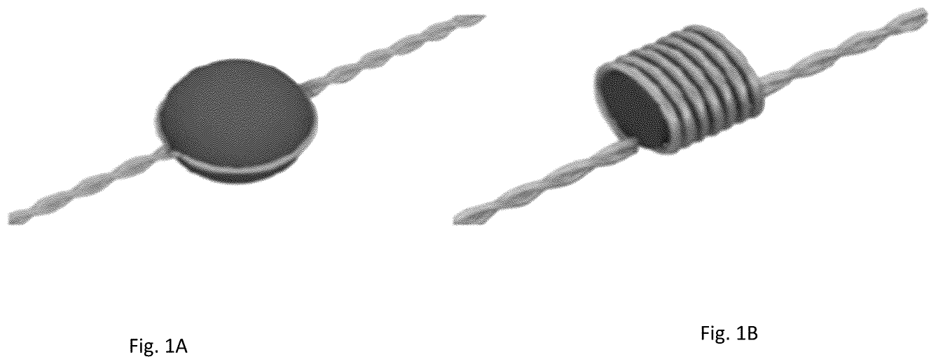

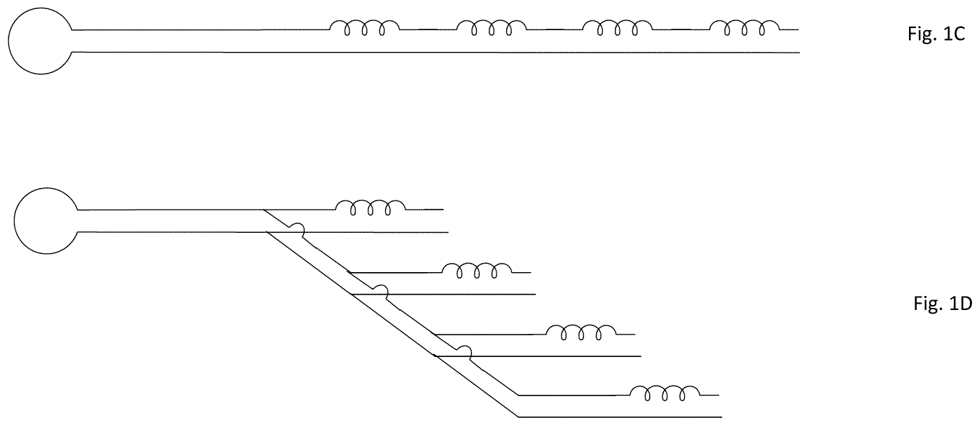

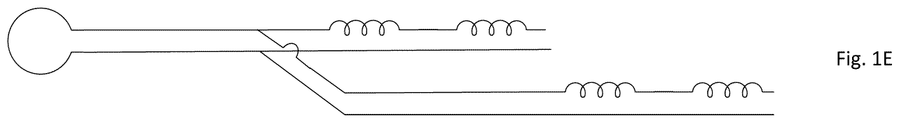

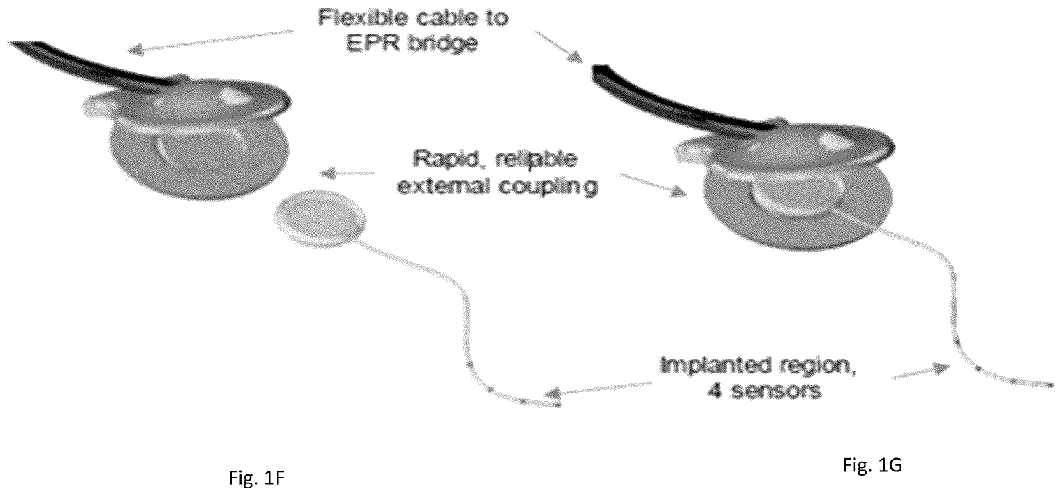

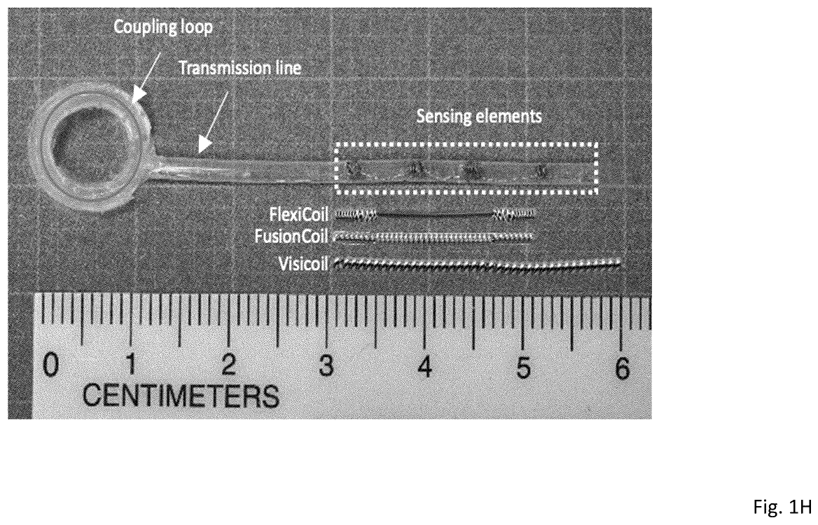

In an effort to overcome the limitations of the initial designs, the inventors have developed a Generation 3 ImR, which is shown in FIG. 1. Specifically, FIG. 1A depicts a first (Generation 2) oxygen sensor used in a deep tissue electron paramagnetic resonance system in accordance with an illustrative embodiment. FIG. 1B depicts a second (Generation 3) oxygen sensor used in a deep tissue electron paramagnetic resonance system in accordance with an illustrative embodiment. As shown, in the proposed device of FIG. 1B, the inventors have replaced the series of single-turn sensing loops in the device of FIG. 1A with multiple-axial turn coils connected in parallel. In an illustrative embodiment, each of the multiple-axial turn coils is open-ended with a twisted pair wire tail of variable length. This allows the EPR phase of each sensor to be independently adjusted by changing the length of twisted pair wire tail at the end of each coil. Additionally, a standing wave can develop in the implant structure along with resonances so the overall length of the implant and the placement of the oxygen sensor coil along that length can have a substantial impact in signal strength. As a result, a tuning process can be implemented to optimize signal strength through placement of the coil. A parameter in this tuning process is that the tail end of the twisted pair wire can be open or closed (i.e., a continuous or broken wire structure).

In an illustrative embodiment, the multiple-axial turn coils that form the sensors can be connected to one another in series, in parallel, or via a combination of series and parallel connections. For example, FIG. 1C depicts a plurality of sensors connected in series in accordance with an illustrative embodiment. FIG. 1D depicts a plurality of sensors connected in parallel in accordance with an illustrative embodiment. FIG. 1E depicts a first subset of sensors connected in series, a second subset of sensors connected in series, and a parallel connection between the first subset and the second subset in accordance with an illustrative embodiment. Any of these various forms of connection (i.e., series, parallel, or a combination of series and parallel) can be used to connect the sensors of the proposed system. Additionally, although four sensors are depicted in the embodiments shown, it is to be understood that a different number of sensors may be used in alternative embodiments such as 1, 2, 3, 5, 8, etc.

With respect to optical placement of the sensing coils in the transmission line, in an open-ended transmission line, the voltage is maximum and radio frequency (RF) current is minimum at the open-end. The incident power is therefore reflected from the open-end. Then the incident and reflected waves form a standing wave such that every half-wavelength (λ/2) along its length from the open-end, the current maximum occurs. Current minimums occur at a distance of λ/4 away from the current maximum locations.

In one embodiment, several sensing coils can be placed symmetrically about the first current maxima from the transmission line open-end. This ensures maximum possible RF current in the sensing coils, and hence maximum RF magnetic field B1. It is noted that increasing transmission line length by multiples of the full-wave length X will not alter the location of the first occurrence of the current maxima from the open-end.

If the coils are in series, then from the location of the last sensing coil, there is an extended length of transmission line length called tail-length. The tail-length of the transmission line at the distal end, and can be iteratively trimmed for optimum EPR signal reception based on testing/experimentation. This iterative trimming of the tail-length optimally locates all the sensing coils from the open-end of the transmission line. If the sensing coils are in parallel configuration, each coil has its own tail-length for optimal location of each coil from its respective tail-length.

In another illustrative embodiment, each of the sensing micro-coils (e.g., ˜0.7 mm in diameter and 1 mm in length in one embodiment), is loaded with O₂ sensitive LiPc microcrystals in liquid PDMS, which rapidly equilibrates with O₂. This LiPc-PDMS mixture is then loaded into the sensing loops of the twisted pair wire structure of the ImR and cured. The entire ImR assembly is then over-molded with PDMS, and the length of the twisted pair transmission line is covered within a thin-walled PTFE tube, which can thereafter remain a part of the ImR assembly. Loop orientation dependence was effectively eliminated with the new ImR depicted in FIG. 1B.

FIG. 1F depicts a sensor system and a coupling mechanism sized to receive a portion of the sensor system in accordance with an illustrative embodiment. FIG. 1G depicts the sensor system mounted to the coupling mechanism in accordance with an illustrative embodiment. In the embodiment shown, the implanted region of the sensor system includes 4 oxygen sensors. In alternative embodiments, the implanted region of the sensor system can include fewer (e.g., 3) or additional (e.g., 5, 6, 8, etc.) oxygen sensors. As shown, the coupling mechanism includes a clamshell that is sized to receive the sensor system. The coupling mechanism includes a flexible cable that connects to an EPR bridge such that oximetry can be performed.

Still referring to FIGS. 1F and 1G, a coupling loop that extends from the implanted part of the structure is placed (under direct visualization) in the clamshell coupling mechanism device, which contains a bridge coupling loop connected to a spectrometer via the flexible radio frequency (RF) cable. Inside the clip the two loops are precisely inductively coupled, with minimal operator interaction. This clip-on structure requires no expertise for placement because the bridge coupling loop has a mechanical mating feature (i.e., the clip, plus the well to hold the two loops in place securely and relative to each other at all times) that promotes proper placement of the two loops throughout the measurement session, regardless of motion. If the loops are not initially placed properly, the clip of the external resonator will not close, and a warning message will be given to correct the placement. The coupled loops are fed with a variable series capacitor, which automatically adjusts the effective coupling of RF energy into the resonator to ensure critical coupling to the ImR. Between measurements, the ImR loop will reside on the surface of the body in a small dressing on the surface where it can be visualized and coupled, making it suitable for use at all potential cancer sites.

FIG. 1H depicts a prototype of the proposed sensing system compared to other common fiducials in accordance with an illustrative embodiment. As shown, the coupling loop and part of transmission line remain external to the body surface. The remaining line and sensor portion (dashed box) are implanted. In an alternative implementation, the transmission line and coupling loop would remain in a body cavity until the time of measurement when the coupling loop would be extract, such as with cervical cancer. The sensor part shown is 1.2 mm diam.×25 mm long with 4 sensors. The flexible transmission line can be any length and the number of sensors (e.g., each 6 mm apart) can be increased as desired. In alternative embodiments, different dimensions, number of sensors, sensor spacing, etc. may be used.

In another illustrative embodiment, using the proposed device, the EPR signal intensity is proportional to a microwave magnetic field B1. As discussed, the proposed implantable resonator design is a micro-coil structure, having multiple turns around each discrete LiPc sensor (4 sensors in the example case). The increase in number of turns increases the resulting B1 inside the micro-coil for the same power level. It is known that the continuous wave EPR signal intensity is related to B1 as shown in Equation 1 below:

$${V}_{s} = {χ}^{″} η Q \sqrt{{PZ}_{0}} . \qquad Equation 1$$

In Equation 1, Vₛ is the signal voltage at the end of the transmission line connected to the resonator, X″ is the imaginary component of the effective RF susceptibility, Q is the loaded quality factor of the resonator, Z₀ is the characteristic impedance of the transmission line, P is the microwave power to the resonator produced by the external microwave source, and η is the resonator efficiency B1/\sqrt{P}. The B1 field is proportional to the current and the number of turns in each of the sensing coil loops. The coil current is proportional to the maximum applied power, which has an upper limit determined by power-induced broadening of the EPR linewidth. However, the B1 field can be increased in the coil by increasing the number of turns such that the ˜0.7 mm diameter LiPc sample is completely embedded inside the micro-coil. The Q of the resonator is only slightly above unity because of the high resistance MP35N wire used.

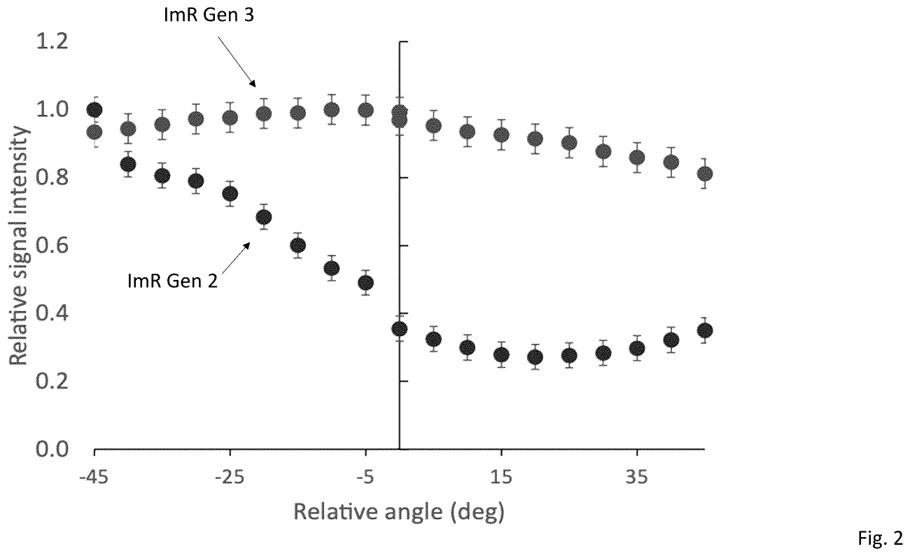

With the increased number of turns, the resonator efficiency is improved, increasing EPR signal detection for a given number of spins. In addition, due to the orientation of the microwave magnetic field (B1) in the axial micro-coils, the proposed Generation 3 ImR yielded a maximal signal intensity regardless of ImR orientation along its long y-axis, given that B1 would remain orthogonal to the z-axis main magnetic field (B0). As shown in FIG. 1A, the Generation 2 ImR required the single loops to be placed facing up along the x-axis to ensure orthogonality to the B0 to achieve maximum signal intensity. Conversely, in the proposed device shown in FIG. 1B, the 360-degrees of rotational freedom about the longitudinal or y-axis of the ImR is highly valuable for in vivo experiments and ultimately clinical use because the ImR is able to rotate during the implantation procedure. In addition, it would be challenging and time-consuming to interrogate and optimize the orientation of the ImR inside the patient for maximum signal intensity. FIG. 2 depicts relative signal intensity of EPR spectra from a Generation 2 sensor system and the proposed sensor system in accordance with an illustrative embodiment. The graph of FIG. 2 further illustrates how the proposed system effectively eliminates loop orientation dependence.

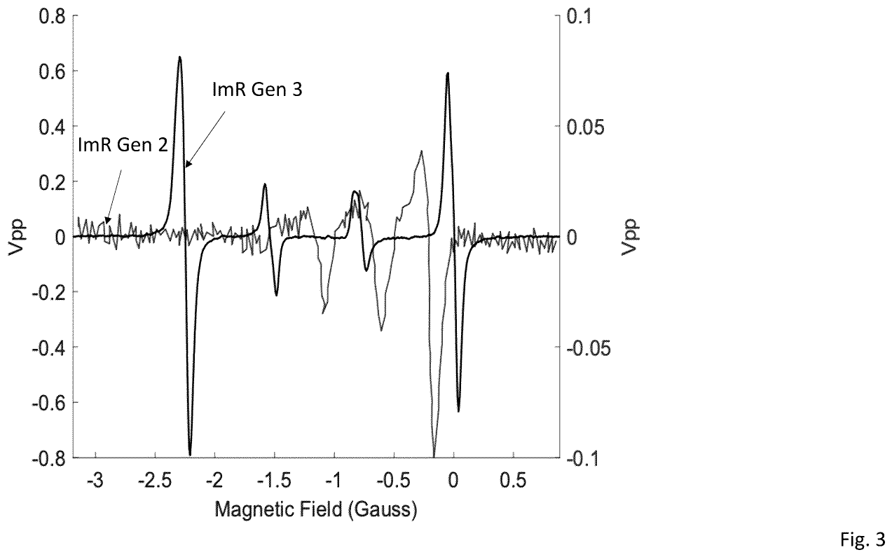

Furthermore, connecting the micro-coils to the transmission line in parallel helps to avoid the reduction in signal intensity across the coils observed in the serial configuration. FIG. 3 depicts a one-minute EPR spectra from a Generation 2 sensor system and the proposed Generation 3 micro-coil sensor system in accordance with an illustrative embodiment. The parallel configuration removed constraints on the number of turns per coil and on the separation between adjacent coils and imposed by proximity to null points on the standing waves of current across the device, as shown by the diminished amplitude in the middle sensors on FIG. 3. The wavelength of the standing waves in the twisted pair transmission line at the working EPR frequency (1145 MHz) was calculated to be 13 cm, based on time delay measurements using a 2-channel network analyzer. This resulted in a velocity factor of 0.5. This latest modification enables freedom to space the ImR sensors as required such that they are located in the hypoxic regions within a tumor along the implantation line, without large drops in signal intensity. The new approach implemented in the Generation 3 ImR thus makes it more versatile, reliable, and promising for clinical pO₂ measurements after implantation in deep tissue due to its increased sensitivity.

In an illustrative embodiment, the system described herein can be used to obtain independent O₂ measurements from each sensing probe within the ImR. Although, in principle there is no maximum number of sensors, initially each ImR can contain 4 sensors in one embodiment. For best spatial resolution, the gradient should be oriented along the principal axis of the ImR. This cannot be assured through implantation, so a three-axis gradient coil system can be used. The strength of the gradient required to resolve a spin probe of 0.1 mT linewidth (LiPc in room air) over 7 mm space between sensors is 14.3 mT/m using a parallel coil Anderson design. Considering future applications in humans, the inventors plan to construct a 90 mT/m gradients system.

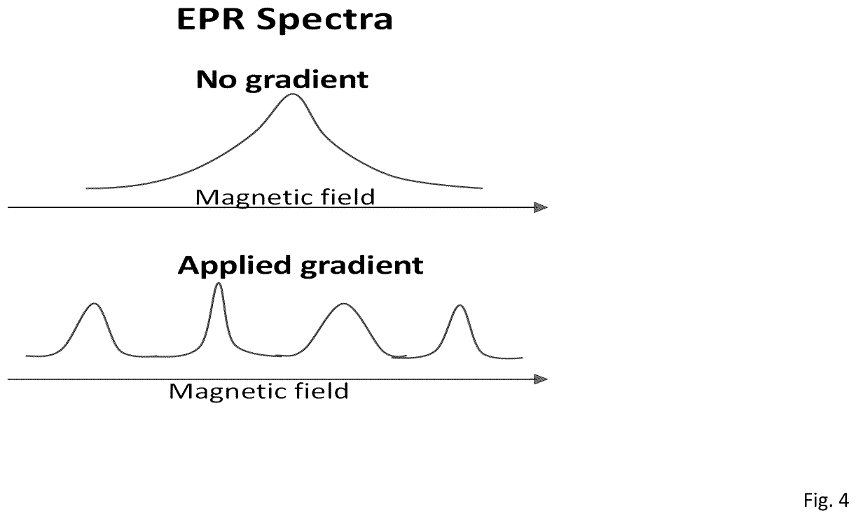

FIG. 4 illustrates how the spatially resolved O₂ measurements from a four-point O₂ probe will be obtained in accordance with an illustrative embodiment. After the proposed modification, i.e., applying 3D gradients (bottom of FIG. 4), the result will be four individual spectra conveying definitive information on the O₂ content and position of each sensor. A phantom can be constructed to test the functionality in resolving disparate O₂ concentrations in the vicinity of spatially separated probes. The phantom can include four 1.6 mm OD×4 mm tubes filled with LiPc equilibrated with 0%, 1%, 2% and 4% pO₂ in nitrogen gas. The phantom can also include 54×50×50 mm pieces of Plexiglas attached with a 48 mm long dowel with an underlying compartment that mimics tissue lossiness at the operational frequency.

The functionality of the complete system can be evaluated by placing one or more ImRs in a tissue equivalent phantom with two separate compartments equilibrated at 0% and 4% pO₂, respectively. Gradient broadening can be accounted for using a calibration based method of broadening versus gradient strength, a filtered back-projection reconstruction algorithm, single-point imaging, or constant-time spectral spatial imaging. The best way to reconstruct the EPR O₂ measurements will be determined in the phantom studies. Criteria to select the gradient coils include the coils' ability to resolve the individual sensing points within the ImR, and the power required to drive the gradients, and ergonomic factors (e.g., comfort, safety, efficiency, and acceptability for the patient and clinical staff, including sufficient automation of the gradient fields). Other criteria can include adequacy of the signal to noise ratio (SNR), the rapidity and convenience for the clinician and the patient, and compatibility with the environment and physical constraints. Using results from preclinical in vivo tests coils can be designed for clinical studies using a phantom that accurately simulates the dimensions and signal loss of the human body.

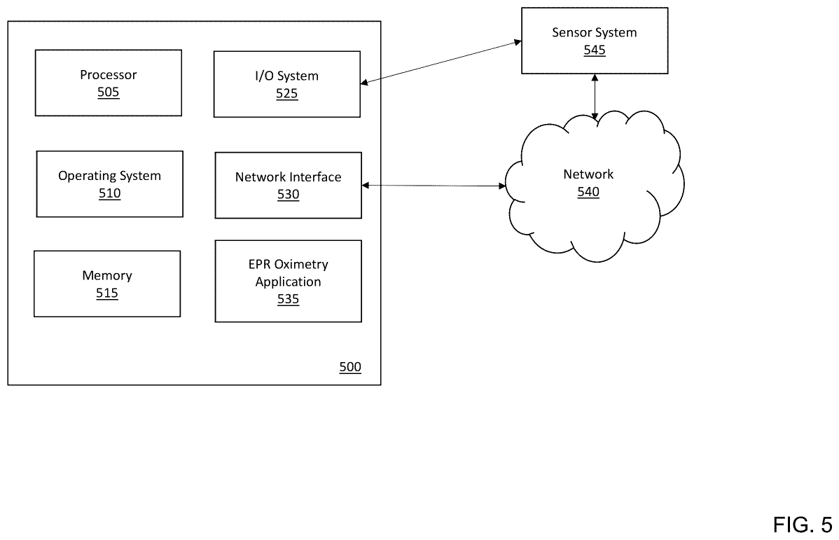

In an illustrative embodiment, any of the operations described herein can be performed in part or in whole by a computing system. FIG. 5 is a block diagram of a computing system 500 for a deep tissue electron paramagnetic resonance system in accordance with an illustrative embodiment. In one embodiment, the computing system 500 can be part of a spectrometer, electron paramagnetic resonance oximetry device, resonator system, etc. Alternatively, the computing system 500 can be part of a laptop computer, a desktop computer, a smartphone, or other device that interacts with another device in the system. The computing system 500 includes a processor 505, an operating system 510, a memory 515, an I/O system 525, a network interface 530, and an EPR oximetry application 535. In alternative embodiments, the computing system 500 may include fewer, additional, and/or different components. The components of the computing system 500 communicate with one another via one or more buses or any other interconnect system.

The processor 505 can be any type of computer processor known in the art, and can include a plurality of processors and/or a plurality of processing cores. The processor 505 can include a controller, a microcontroller, an audio processor, a graphics processing unit, a hardware accelerator, a digital signal processor, etc. Additionally, the processor 505 may be implemented as a complex instruction set computer processor, a reduced instruction set computer processor, an x86 instruction set computer processor, etc. The processor 505 is used to run the operating system 510, which can be any type of operating system.

The operating system 510 is stored in the memory 515, which is also used to store programs, network and communications data, peripheral component data, oximetry data, patient information, the EPR oximetry application 535, and other operating instructions. The memory 515 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc.

The I/O system 525 is the framework which enables users and peripheral devices to interact with the computing system 500. The I/O system 525 can include a mouse, a keyboard, one or more displays, a speaker, a microphone, etc. that allow the user to interact with and control the computing system 500. The I/O system 525 also includes circuitry and a bus structure to interface with peripheral computing devices such as power sources, USB devices, peripheral component interconnect express (PCIe) devices, serial advanced technology attachment (SATA) devices, high definition multimedia interface (HDMI) devices, proprietary connection devices, etc. In an illustrative embodiment, the I/O system 525 is configured to receive inputs and operating instructions from the user.

In an illustrative embodiment, the I/O system 525 is also in communication with a sensor system 545, which can be any of the sensor systems described herein. For example, the sensor system 545 can include an implant portion having a plurality of multiple-axial turn coils connected in parallel, where each coil includes an oxygen sensor. Each of the multiple-axial turn coils can be open-ended with a twisted pair wire tail of variable length. To form the sensors, each of the sensing micro-coils can be loaded with O₂ sensitive LiPc microcrystals in liquid PDMS, and this mixture is loaded into the sensing loops of the twisted pair wire structure of the ImR and cured using any curing technique known in the art. The ImR assembly can also be over-molded with PDMS, and the length of the twisted pair transmission line is covered within a thin-walled PTFE tube, as described herein.

Connected to the implant portion of the sensor system 545 is a coupling loop, and the coupling loop is sized to mate with a coupling mechanism of the sensory system 545. In an illustrative embodiment, the coupling mechanism includes a clamshell design that is sized to clamp around the coupling loop such that oximetry can be performed. The coupling mechanism of the sensor system 545 also includes a flexible cable that connects to the computing system 500 and/or another device.

The network interface 530 includes transceiver circuitry that allows the computing system to transmit and receive data to/from other devices such as remote computing systems, servers, websites, etc. The network interface 530 enables communication through the network 540, which can be in the form of one or more communication networks and devices. For example, the network 540 can include a cable network, a fiber network, a cellular network, a wi-fi network, a landline telephone network, a microwave network, a satellite network, etc. and any devices/programs accessible through such networks. The network interface 530 also includes circuitry to allow device-to-device communication such as Bluetooth® communication.

The EPR oximetry application 535 includes hardware and/or software, and is configured to perform any of the operations described herein. Software of the EPR oximetry application 535 can be stored in the memory 515 as computer-readable instructions. As an example, the EPR oximetry application 535 can include computer-readable instructions to perform device tuning, generate an error message if the coupling loop is not properly seated in the coupling mechanism, determining and analyzing magnetic fields, determining oxygen levels, determining standing wave characteristics, constructing phantoms, determining an appropriate amount of radiation and/or a time at which to perform radiation treatment based on oxygen readings, etc.

Thus, described herein is a next generation sensor system that provides spatially distributed, repeatable, and reliable O₂ measurements at any medically relevant depth under clinically useful conditions to optimize therapeutic delivery of radiation. The system provides knowledge about hypoxia levels from interventions and treatment over the course of therapy, which can be used to optimize the timing of radiotherapy fractions to be delivered. This will allow physicians to evaluate and demonstrate, for the first time, improved outcomes that should result from delivering treatment fractions at the time when oxygenation is improved in hypoxic tumor regions.

The system described herein has numerous advantages over existing systems. For example, the proposed system replaces the single-turn sensing loops used in traditional systems with multiple-turn coils. An advantage of multiple-turn coils is a signal increase in proportion to the number of turns around the paramagnetic oxygen probes. Additionally, single-turn sensing loops are oriented perpendicular to the axis of implant, whereas multiple-turn coils are oriented axially. The axial orientation provides rotational freedom about the longitudinal or y-axis of the ImR without sacrificing signal intensity. Also, sensors previously connected in series are now connected in parallel. The parallel configuration removes constraints on numbers of turns per coil and distance between sensors to avoid null points in the standing waves of current down the transmission line. Individual coils can be made and connected as required for a given application. Further, in the proposed system, the open end was moved from the coupling loop side to after the end of the last coil. A twisted pair tail was also added after the sensors. Using these features, the EPR phase of each sensor can be independently adjusted by changing the length of twisted pair wire tail at the end of each coil (each coil has a tail if connected in parallel).

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. An oxygen sensing system comprising:
a coupling loop that connects to an electron paramagnetic resonance (EPR) bridge;
a cable connected to the coupling loop, wherein the cable acts as a transmission line; and
a plurality of oxygen sensors formed on the cable, wherein each of the oxygen sensors comprises a multi-turn coil.

2. The system of claim 1, wherein the plurality of oxygen sensors are connected in parallel.

3. The system of claim 1, wherein the cable comprises a twisted pair wire cable.

4. The system of claim 3, wherein each oxygen sensor includes a twisted pair tail that extends from the oxygen sensor.

5. The system of claim 4, wherein the twisted pair tail varies in length for each of the oxygen sensors.

6. The system of claim 1, wherein each multi-turn coil includes microcrystals that are coated with lithium phthalocyanine (LiPc).

7. The system of claim 1, further comprising a polydimethylsiloxane (PDMS) layer that is molded over the cable and the plurality of oxygen sensors.

8. The system of claim 1, further comprising a polytetrafluoroethylene (PTFE) tube that covers the plurality of oxygen sensors and at least a portion of the cable.

9. The system of claim 8, wherein the portion of the cable and the plurality of oxygen sensors form an implant portion of the system, and wherein the PTFE tube covers the implant portion.

10. The system of claim 1, wherein the multi-turn coil of each oxygen sensor is oriented axially relative to the cable.

11. The system of claim 1, further comprising the EPR bridge, wherein a clamp mechanism is mounted to an end of the EPR bridge, and wherein the clamp mechanism is sized to receive the coupling loop.

12. The system of claim 11, wherein the clamp mechanism comprises a clamshell clamp that closes around the coupling loop.

13. A method of forming an oxygen sensing system, the method comprising:
forming a coupling loop that is sized to connect to an electron paramagnetic resonance (EPR) bridge;
mounting a cable to the coupling loop, wherein the cable acts as a transmission line; and
forming a plurality of oxygen sensors on the cable, wherein each of the oxygen sensors comprises a multi-turn coil.

14. The method of claim 13, wherein the plurality of oxygen sensors are formed on the cable such that the oxygen sensors are connected in parallel.

15. The method of claim 13, wherein the plurality of oxygen sensors are formed such that each oxygen sensor includes a twisted pair tail that extends from the oxygen sensor.

16. The method of claim 15, wherein the twisted pair tail varies in length for each oxygen sensor.

17. The method of claim 13, forming a mixture of lithium phthalocyanine (LiPc) microcrystals and polydimethylsiloxane (PDMS) and loading the mixture into the multi-turn coil to form each oxygen sensor in the plurality of oxygen sensors.

18. The method of claim 13, wherein at least a portion of the cable and the plurality of oxygen sensors form an implant portion of the system, and further comprising covering the implant portion with polytetrafluoroethylene (PTFE) tube.

19. The method of claim 13, further comprising mounting a clamp mechanism to the EPR bridge, wherein the clamp mechanism is sized to receive the coupling loop.

20. The method of claim 19, further comprising forming the clamp mechanism as a clamshell clamp.
